(19) United States
(12) Patent Application Publication
(10) Pub. No.: US 2010/0016445 A1
(43) Pub. Date: Jan. 21, 2010

(54) TRANSDERMAL DIETHYLSTILBESTROL FOR TREATING PROSTATE CANCER

(75) Inventor: Tomasz M. Beer, Portland, OR (US)

Correspondence Address: KLARQUIST SPARKMAN, LLP, 121 SW SALMON STREET, SUITE 1600, PORTLAND, OR, 97204, US

(73) Assignee: Oregon Health and Science University

(21) Appl. No.: 12/421,471

(22) Filed: Apr. 9, 2009

Related U.S. Application Data
(63) Continuation-in-part of application No. PCT/US2007/021621, filed on Oct. 9, 2007.
(60) Provisional application No. 60/851,255, filed on Oct. 11, 2006.

(51) Int. Cl. A61K 31/05; A61P 35/00

(52) U.S. Cl. 514/733

(57) ABSTRACT

Methods for treating prostate cancer comprising transdermally administering a therapeutically effective amount of diethylstilbesterol (DES), or a pharmaceutically acceptable salt or complex thereof, to a subject. In one embodiment, transdermally administered DES may be used to treat hot flashes in castrate prostate cancer patients. In another embodiment, transdermal DES may be administered as a therapy while avoiding testosterone surge and clinical tumor flare (for example, for treating symptomatic locally advanced prostate cancer, treating metastatic prostate cancer, or in a subject who is contraindicated for conventional therapy such as ADT therapy, particularly GnRH (gonadotropin-releasing hormone) therapy). In a further embodiment, transdermal DES may be administered to treat osteoporosis in men with prostate cancer. The transdermal DES may be used as a front line hormonal therapy or a second line hormonal therapy for treating prostate cancer.

This application is a continuation-in-part of International Application No. PCT/US2007/021621, filed Oct. 9, 2007, which claims the benefit of U.S. Provisional Application No. 60/851,255, filed Oct. 11, 2006, both of which are incorporated herein by reference in their entireties.

### FIELD

The present disclosure relates to transdermal administration of diethylstilbesterol (DES).

### BACKGROUND

Androgen deprivation therapy (ADT) is the mainstay of management of advanced prostate cancer and recently has been shown to improve survival when administered in earlier stages of the disease. The oncologic benefits of ADT may be partially offset, however, by a reduction in quality of life due to adverse effects. In addition to the well-recognized adverse consequences of ADT, recent evidence suggests that ADT is associated with dyslipidemia, impaired glucose metabolism, adverse body compositional changes, and osteoporosis. Thus, there is a pressing need to develop less toxic forms of ADT.

More specifically, a dramatic increase in the use of androgen deprivation therapy (ADT) in recent years, particularly the expansion of use in earlier stages of prostate cancer, creates an urgent and compelling need to carefully examine the long-term toxicities of ADT and investigate the use of potentially less toxic alternative treatment regimens. Examination of recent practice patterns in prostate cancer reveals a marked departure from previous patterns of ADT use.¹⁻³ Previously used primarily to treat men with advanced metastatic disease, a group whose life expectancy is typically approximately 3 years, ADT is now the most rapidly growing treatment modality for localized prostate cancer⁴ and is routinely used as adjuvant therapy in men at high risk for relapse after either surgery or radiation.⁵⁻⁷ The use of serum PSA for closer monitoring of prostate cancer frequently leads to the early deployment of ADT.⁸ Thus, several forces have fueled an overall increase in the use of ADT as well as a shift in use earlier in the course of the disease: (1) increased incidence of prostate cancer, (2) more vigilant monitoring of disease, (3) new evidence of benefit in the adjuvant and early metastatic settings, and (4) therapeutic enthusiasm in the U.S. medical community. Today, unlike in the past, this therapy is often offered to men whose life expectancy exceeds a decade, who are not symptomatic from their prostate cancer, and whose probability of dying from prostate cancer may be quite low.⁹

Recent years have brought greater appreciation for the potential toxicities of ADT. Accelerated bone loss has been well described,¹⁰ and unfavorable lipid changes that may contribute to premature cardiovascular disease have been recognized.¹¹ Adverse body compositional changes, insulin resistance, and worsening of diabetes control, have also been demonstrated.¹²⁻¹⁵ Other recognized toxicities of commonly used forms of ADT include loss of libido, erectile dysfunction, hot flashes, liver function abnormalities, diarrhea and other gastrointestinal symptoms, pulmonary toxicities, decreased light accommodation, alcohol intolerance, and rash.¹⁶ These toxicities result in reduction in quality of life when ADT is used in asymptomatic patients.¹⁷ The impact of adverse effects on the risk-benefit balance is proportionately greater as ADT is prescribed for men whose risk of harm due to cancer is lower.

A novel approach to this problem is the use of estrogens to induce androgen suppression. In 1941 Huggins and Hodges reported that the oral estrogen diethylstilbesterol (DES) had similar effects on prostate cancer as surgical castration.¹⁸ This observation was confirmed in several randomized trials.¹⁹ Several excellent reviews of oral estrogen in the treatment of prostate cancer are available.²⁰⁻²² However, the role of oral estrogens in the management of prostate cancer has been limited because of an association with thromboembolic toxicity.

The first controlled trials to be carried out with estrogen were the Veterans Administration Cooperative Urological Research Group (VACURG) studies. When compared with orchiectomy, DES therapy was associated with a non-significant trend toward better cancer control, but also a significant increase in cardiovascular morbidity and mortality at the higher doses.²³ Non-cancer related deaths, mostly cardiovascular in origin, were increased by 36% in patients receiving 5 mg of DES per day. Further studies evaluating lower doses of DES reported varying results of testosterone suppression and thromboembolic toxicity, although overall survival with this therapy was similar to other studies of ADT.^{24,45-48}

The combined information from these trials led to adoption of 3 mg per day as the most commonly used DES oral dose for prostate cancer.⁴⁹ However, the thromboembolic toxicity remained a concern, and when GnRH agonists became available, DES rapidly fell out of favor. In 1984, the Leuprolide Study Group published results of a randomized trial of DES 3 mg versus leuprolide (Lupron) in metastatic prostate cancer.⁴⁹ There were no significant differences in suppression of testosterone, overall response rates, and overall survival rates. Patients receiving DES had more frequent painful gynecomastia, nausea and vomiting, edema, and thromboembolism, whereas those in the leuprolide group had more frequent hot flashes. The authors concluded that leuprolide was therapeutically equivalent to DES, with fewer side effects. This trial led to widespread acceptance of leuprolide as initial hormonal management for metastatic prostate cancer.

### SUMMARY

Disclosed herein are methods for treating prostate cancer comprising transdermally administering a therapeutically effective amount of diethylstilbesterol (DES), or a pharmaceutically acceptable salt or complex thereof, to a subject. In one embodiment, transdermally administered DES may be used to treat hot flashes in castrate prostate cancer patients. In another embodiment, transdermal DES may be administered as a therapy while avoiding testosterone surge and clinical tumor flare (for example, for treating symptomatic locally advanced prostate cancer, treating metastatic prostate cancer, or in a subject who is contraindicated for conventional therapy such as ADT therapy, particularly GnRH (gonadotropin-releasing hormone) therapy). In a further embodiment, transdermal DES may be administered to treat osteoporosis in men with prostate cancer. The transdermal DES may be used as a front line hormonal therapy or a second line hormonal therapy for treating prostate cancer.

Also disclosed herein are methods for treating a taxane-resistant disease (for example, a taxane-resistant cancer) comprising transdermally administering a therapeutically effective amount of diethylstilbesterol (DES), or a pharmaceutically acceptable salt or complex thereof, to a subject who has, or is diagnosed with, a taxane-resistant disease. An example of a taxane-resistant disease is a neoplasm that relapses during or after treatment with a taxane (examples include docetaxel, paclitaxel, and various formulations of these, such as polyglutamate paclitaxel (Xyotax, CTI Pharamceuticals, albumin-bound paclitaxel (Abraxis, Abraxane Pharmaceuticals), and other similar formulations of taxanes). The time to relapse after treatment varies from condition to condition and can range from relapse during therapy to relapse that occurs within 1 year of therapy. Examples of taxane-treated disease that become taxane-resistant include prostate cancer, breast cancer, bladder cancer, lung cancer, and ovarian cancer. Such administration may also be in combination (i.e., co-administration) with another chemotherapeutic agent (e.g., a taxane (such as paclitaxel or docetaxel)).

Also disclosed herein are methods for treating skeletal-related complications comprising transdermally administering a therapeutically effective amount of diethylstilbesterol (DES), or a pharmaceutically acceptable salt or complex thereof, to a subject.

The foregoing and other objects, features, and advantages will become more apparent from the following detailed description, which proceeds with reference to the accompanying figures.

### FIGURES

FIG. 1 is a graph depicting the results of the mouse sample analysis by TSQ-ESI-MS.

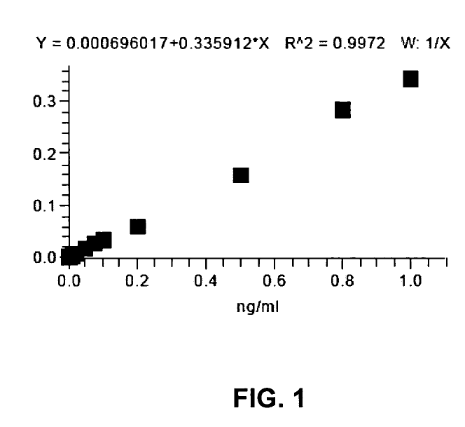

### DETAILED DESCRIPTION

“Administration of” and “administering a” compound should be understood to mean providing a compound, a prodrug of a compound, or a pharmaceutical composition as described herein. The compound or composition can be administered by another person to the subject or it can be self-administered by the subject (e.g., patches).

“Androgen-independent prostate cancer (AIPC)” refers to prostate cancer that progresses despite administration of primary hormonal therapy, such as androgen deprivation therapy.

An “animal” is a living multicellular vertebrate organism, a category that includes, for example, mammals and birds. A “mammal” includes both human and non-human mammals. “Subject” includes both human and animal subjects.

“Dosage” means the amount delivered in vivo to a subject of a compound, a prodrug of a compound, or a pharmaceutical composition as described herein.

“Pharmaceutically acceptable salts” of DES include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002).

“Pharmaceutically acceptable complexes” of the presently disclosed DES include those complexes or coordination compounds formed from metal ions. Such complexes can include a ligand or chelating agent for bonding with DES.

A “pharmaceutical agent” or “drug” refers to a chemical compound or composition capable of inducing a desired therapeutic or prophylactic effect when properly administered to a subject.

“Inhibiting” means that the compounds or pharmaceutical compositions disclosed herein will slow or stop the development of disease symptoms or delay the onset of the disease. For example, “inhibiting” or “treating” refers to inhibiting the full development of a disease or condition, for example, in a subject who is at risk for a disease or condition. In certain embodiments, “treatment” refers to a therapeutic intervention that ameliorates a sign or symptom of a disease or pathological condition after it has begun to develop. As used herein, the term “ameliorating,” with reference to a disease, pathological condition or symptom, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology. By the term “co-administer” is meant that each of at least two compounds be administered during a time frame wherein the respective periods of biological activity overlap. Thus, the term includes sequential as well as coextensive administration of two or more drug compounds.

A “therapeutically effective amount” is an amount effective to reduce or lessen at least one symptom of the disease or condition being treated or to reduce or delay onset of one or more clinical markers or symptoms of the disease or condition. The therapeutically effective amount of an agent will be dependent on the subject being treated, the severity of the affliction, and the manner of administration of the therapeutic composition.

“Transdermal” drug delivery means administration of a drug to the skin surface of an individual so that the drug passes through the skin tissue and into the individual's blood stream, thereby providing a systemic effect. The term “transdermal” is intended to include “transmucosal” drug administration, i.e., administration of a drug to the mucosal (e.g., sublingual, buccal, rectal) surface of an individual so that the drug passes through the mucosal tissue and into the individual's blood stream.

The above term descriptions are provided solely to aid the reader, and should not be construed to have a scope less than that understood by a person of ordinary skill in the art or as limiting the scope of the appended claims.

The singular terms “a,” “an,” and “the” include plural referents unless context clearly indicates otherwise. Similarly, the word “or” is intended to include “and” unless the context clearly indicates otherwise. The word “comprises” indicates “includes.” It is further to be understood that all molecular weight or molecular mass values given for compounds are approximate, and are provided for description. Although methods and materials similar or equivalent to those described herein can be used in the practice or testing of this disclosure, suitable methods and materials are described below. In addition, the materials, methods, and examples are illustrative only and not intended to be limiting. All chemical compounds disclosed herein include both the (+) and (−) stereoisomers (as well as either the (+) or (−) stereoisomer), and any tautomers thereof.

An analog is a molecule that differs in chemical structure from a parent compound, for example a homolog (differing by an increment in the chemical structure, such as a difference in the length of an alkyl chain), a molecular fragment, a structure that differs by one or more functional groups, or a change in ionization. Structural analogs are often found using quantitative structure activity relationships (QSAR), with techniques such as those disclosed in Remington: The Science and Practice of Pharmacology, 19^{th} Edition (1995), chapter 28. A derivative is a biologically active molecule derived from the base structure.

As described above, disclosed herein are methods for transdermally administering DES to treat or inhibit various diseases, conditions, indications, or atypical biological functions. DES (also referred to as diethylstilbestrol or 4-[4-(4-hydroxyphenyl)hex-3-en-3-yl]phenol) is distinct from estradiol in its structure and, in part, in the mechanisms of its antineoplastic activity. For example, preclinical data demonstrates that DES modulates tubulin expression in prostate carcinoma cells and by doing so increases the taxane sensitivity of prostate cancer cell lines (see Montgomery et al., “Estrogen Effects on Tubulin Expression and Taxane Mediated Cytotoxicity in Prostate Cancer Cells,” The Prostate 65:141-150 (2005), which is incorporated herein by reference in its entirety). This effect does not occur with estradiol.

The DES dosage may vary depending upon the specific conditions and patients undergoing treatment. The therapeutically effective dosage of the compound can be provided as repeated doses within a prolonged prophylaxis or treatment regimen that will yield clinically significant results to alleviate one or more symptoms or detectable conditions associated with a targeted disease or condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the compound (for example, amounts that are effective to elicit a desired immune response or alleviate one or more symptoms of a targeted disease). In alternative embodiments, an effective amount or effective dose of the compound may simply inhibit or enhance one or more selected biological activities correlated with a disease or condition, as set forth herein, for either therapeutic or diagnostic purposes.

The actual dosage of the compound will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the compound for eliciting the desired activity or biological response in the subject. Dosage regimens can be adjusted to provide an optimum prophylactic or therapeutic response. A therapeutically effective amount is also one in which any toxic or detrimental side effects of the compound and/or other biologically active agent is outweighed in clinical terms by therapeutically beneficial effects. For example, the DES may be administered in a dose of at least about 0.1, particularly about 0.2, more particularly about 0.5, most particularly about 1.0, and even more particularly about 5.0 mg/day. The maximum dosage, for example, may be about 25, particularly about 10, more particularly about 5.0, and most particularly about 3.0 mg/day.

The dose may be a single dose per day, it may be divided into at least two unit dosages for administration over a 24-hour period, or it may be a single continuous dose for a longer period of time, such as 3 days to 10 weeks or 1-10 weeks. In the example of transdermal administration via a patch(es) that is described below in more detail, the patch(es) may be applied daily, every three days, every seven days, or at other suitable intervals. It should also be recognized that the amount of DES included in a single patch should be sufficient to deliver the dosages described above. For example, a patch may contain 8 mg of DES that transdermally delivers on a daily basis a certain fraction (for example, 0.2 mg) of the 8 mg. Treatment may be continued as long as necessary to achieve the desired results. For instance, treatment may continue for about 3 or 4 weeks up to about 12-24 months, and possibly indefinitely.

The treatment disclosed herein involves administering to a patient in need of such treatment a pharmaceutical composition that includes a pharmaceutically acceptable carrier and a therapeutically-effective amount of DES. The compounds or pharmaceutical compositions are administered transdermally since oral administration of DES to a male subject is associated with increased risk of thromboembolic complications. Transdermal administration may be accomplished via transdermal patches, lotions, creams, gels, pastes, sprays, ointments, eye drops, nose drops, ear drops, suppositories and/or similar transdermal administration techniques.

For application topically to the skin, the DES-containing pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound (e.g., a synthetic block copolymer of ethylene oxide and propylene oxide), emulsifying wax, water, myristic acid salt or ester, and lecithin. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water

In general, DES transdermal delivery patch systems may include a skin-adhering substrate and a DES-containing formulation coated or incorporated into the substrate that can be released to the skin over a prolonged period (e.g., from 1 to 30 days). The patches typically are applied to a predetermined area of the skin. “Predetermined area” of skin or mucosal tissue refers to the area of skin or mucosal tissue through which a drug-enhancer formulation is delivered, and includes a defined area of intact unbroken living skin or mucosal tissue. That area will usually be in the range of about 5 cm² to about 200 cm², more usually in the range of about 5 cm² to about 100 cm², preferably in the range of about 20 cm² to about 60 cm². However, it will be appreciated by those skilled in the art of drug delivery that the area of skin or mucosal tissue through which the drug is administered may vary significantly, depending on patch configuration, dose, and the like.

Illustrative patches generally include a porous membrane, a thin, flexible, polymer film that covers the adhesive; a drug (i.e., DES); an adhesive, usually silicone; and a release liner, the film that is removed prior to application. The DES may exist as either a liquid or a gel in a drug reservoir, or it may be dispersed in a polymeric material. In other embodiments, the patch may include a largely moisture impermeable and effective-ingredient-impermeable backing layer, an effective ingredient containing adhesive layer and a removable protective layer. The DES may be completely dissolved in the adhesive layer. One example of an adhesive layer comprises DES and an adhesive matrix based on a silicone polymer, a polyisobutylene polymer (PIB), a polyacrylate polymer or a styrene block copolymer with butadiene or isoprene (SBS or SIS).

Another example of a transdermal patch includes (proceeding from the visible surface to the surface attached to the skin) a translucent polyethylene film and an acrylate adhesive matrix containing DES. A protective liner is attached to the adhesive surface and must be removed before the system can be used. U.S. Pat. No. 5,223,261 (Nelson et al.) describes the transdermal patch systems. The system can also include (in addition to DES and an acrylic adhesive) a skin penetration enhancer combination that includes fatty acid esters such as isopropyl myristate, glyceryl monolaurate, and ethyl oleate. The acrylic adhesive may be an acrylic polymer comprising at least about 91 to 98 percent by weight of a hydrophobic monomeric acrylic or methacrylic acid ester of an alkyl alcohol based on the weight of all monomers in the polymer or an acrylic copolymer comprising (i) about 60 to 80 percent by weight of a hydrophobic monomeric acrylic or methacrylic acid ester of an alkyl alcohol based on the weight of all of the monomers in the copolymer; (ii) about 4 to 9 percent by weight based on the weight of all of the monomers in the copolymer of a reinforcing monomer selected from acrylic acid, methacrylic acid, an alkyl acrylate or methacrylate containing 1 to 3 carbon atoms in the alkyl group, acrylamide, methacrylamide, a lower diacetone acrylamide, and a N-vinyl-2-pyrrolidone; and (iii) about 15 to 35 percent by weight of vinyl acetate based on the weight of all of the monomers in the copolymer. The DES may be present in an amount of about 0.2 to 12 weight percent of the total weight of the adhesive coating.

Another illustrative transdermal patch system includes five layers. Proceeding from the visible surface toward the surface attached to the skin, these layers are (1) a transparent polyester backing film, (2) a drug reservoir of DES, (3) an ethylene vinyl acetate copolymer release-controlling membrane, (4) an adhesive formulation of light mineral oil and polyisobutylene, and (5) a protective liner of siliconized polyethylene terephthalate film attached to the adhesive surface for removal prior to use.

An advantage of the methods described herein relates to the shifting pattern in ADT use. The potential advantages of transdermal DES-based therapy accrue over time and may be barely perceptible in patients whose cancer-related life expectancy is short. On the other hand, these advantages may be decisive when therapy occurs early in the life of men expected to live a decade or longer.

The most widely recognized action of estrogen in prostate cancer is its impact on the hypothalamic-pituitary-gonadal axis. Estrogen inhibits gonadotropin-releasing hormone (GnRH) and leutinizing hormone (LH) released via negative feedback loops at both the hypothalamus and pituitary. This, in turn, inhibits testosterone release.²²⁻²⁶

Estrogen also has antitumor effects independent of this systemic pathway, mediated by the direct action of estrogen on prostate cancer cells. There is in vitro data for antimitotic and pro-apoptotic effects of estrogenic compounds.²⁷⁻³² Many of these effects are independent of the estrogen receptor and may explain, in part, the activity of estrogens in androgen insensitive prostate cancer.³³ For example, DES induces apoptosis directly even in androgen insensitive prostate cancer cells, and this effect is not dependent on the presence of estrogen receptors.²⁷ In an animal model, 17β-estradiol supplementation was shown to inhibit prostate cancer growth by mechanisms that were independent of androgen action.³⁴ Similarly, a metabolite of estrogen, 2-methoxyestradiol, induces apoptosis in both androgen-dependent and androgen-independent prostate cancer cell lines,³⁵⁻³⁷ and has been shown to inhibit angiogenesis by disrupting microtubules and dysregulating hypoxia-inducible factor-1 (HIF).³⁸ The preliminary results of a phase II study of 2-methoxyestradiol in hormone refractory prostate cancer show that this agent is well tolerated; efficacy data are not yet available.³⁹

To date, two estrogen receptor (ER) subtypes have been cloned, ER alpha and ER beta. The binding affinity of ER alpha for estradiol is higher than that of ER beta.⁴⁰ ER mRNA or ER immunoreactivity of both subtypes has been demonstrated in the prostate,⁴¹ but their function and possible role in prostate cancer initiation and progression are not understood.²⁹ Progressive loss of ER beta expression has been found in prostatic hyperplasia and, to a greater extent, in invasive cancer.⁴² Prostate tumors that retained ER beta, as measured by immunohistochemistry, were associated with a greater rate of relapse than ER-negative tumors.⁴² However, when ER beta was measured by RT-PCR, no significant associations between the expression of ER beta and prostate tumor type were found.⁴³ The role of ER beta in regulating the growth of different tumors is complex, and the link between ER beta and prostate cancer is not yet clear. The presence of functional ER beta may represent a potential therapeutic target.⁴⁴

Thus, ER signaling may play a dual role in prostate cancer progression and therapy. At physiologic doses estrogen may be involved with prostate cancer development, whereas, in more aggressive prostate cancer, intact ER signaling pathways may betargeted for achieving therapeutic benefits.

Recent clinical data strongly suggest that administration of estrogen via the parenteral route may circumvent the thromboembolic cascade of events associated with oral administration of these drugs.^{25,26,45,50-53} Scandinavian investigators have extensively studied the use of intramuscular estrogens, their effects on coagulation proteins, and associated toxicities.^{26,54-57} In an initial phase I trial, intramuscular polyoestradiol phosphate (PEP) did not cause any cardiovascular or thromboembolic complications in 12 patients with newly diagnosed prostate cancer treated for a median of 12.9 months.⁵⁵

Subsequently, the Scandinavian Prostate Cancer Group compared the efficacy and safety of PEP to orchiectomy or combined androgen blockade in 915 men with newly diagnosed metastatic hormone-naïve prostate cancer. With a median follow-up of 27 months, there was no difference in overall, prostate cancer, or cardiovascular mortality between groups.²⁵ There was also no difference in time to biochemical or clinical progression of prostate cancer. Non-fatal cardiovascular events were more frequent in the PEP group (3.7% versus 1.1% for ischemic heart disease, p=0.009; and 4.4% versus 2.0% for heart failure, p=0.035). However, patients randomized to PEP were more likely to have cardiovascular disease at baseline prior to study entry (17.1% versus 14.5%, p value not reported) and this imbalance in baseline risk could have contributed to the observed differences in cardiovascular morbidity. Importantly, the incidence of cardiovascular events encountered in the PEP group was much lower than that registered in earlier studies of DES and was not associated with excess cardiovascular mortality. PEP therapy was also associated with more gynecomastia and fewer hot flashes than conventional ADT. This landmark study was the first to demonstrate that a parenterally administered estrogenic formulation could be both safe and effective for the treatment of hormone-naïve prostate cancer. This depot estradiol formulation is not available in the United States.

More recently, the transdermal administration of estradiol as a prostate cancer treatment has been examined in two small studies.^{58,59} Ockrim and colleagues evaluated transdermal estradiol in 20 patients with advanced hormone-naïve prostate cancer followed for a median of 10 months. This therapy resulted in castrate testosterone levels and tumor responses in all 20 patients.⁵⁸ No cardiovascular or thromboembolic complications were seen.

A second trial group recently reported results of a phase II trial of transdermal estrogen in patients with androgen-independent prostate cancer.⁵⁹ Three of 24 patients had a confirmed PSA reduction >50%, and 7 patients achieved a PSA reduction >25%. The total serum testosterone level remained in the anorchid range in all patients during treatment. No thromboembolic complications occurred. This study included measures of coagulation, inflammation, lipids, bone turnover, and bone density, which are discussed below.

The mechanism of estrogen-mediated coagulopathy has been ascribed to multiple abnormalities. Oral estrogen increases factor VIII activity, induces prothrombin activation, and is associated with a decrease in fibrinogen and increased resistance to activated protein C (APC).^{26,55,60-62} Oral estrogen undergoes significant metabolism in the liver, i.e. first pass effect, which may contribute to unfavorable changes in coagulation factors.^{54,63-69} In addition to increasing factor VIII activity, estrogens induce the formation of specific liver-synthesized proteins, including factor VII.²⁶

In contrast, the absence of effects on coagulation factors may explain the apparent reduction in cardiovascular and thromboembolic toxicity seen when estrogens are parenterally administered.⁷⁰⁻⁷² Consistent with avoidance of first-pass metabolism, parenteral estrogen is not associated with induction of hepatic protein synthesis of coagulation factors.⁷³⁻⁷⁶ Parenteral administration is associated with a different pattern of estradiol metabolism that results in higher blood estradiol to estrone ratio.⁷⁷

In the Scandinavian studies, PEP caused no change in any of the coagulation factors, including factor VII, with the exception of a significant decrease in antithrombin III.⁵⁵ Fibrinogen levels were unchanged.⁵⁵ Activated protein C (APC) resistance was not measured. In a phase II study, transdermal estradiol did not affect factor VIII activity, F1.2, or resistance to APC. There was a modest reduction in the level of protein S that is unlikely to be clinically significant as levels remained in the normal range for all patients.⁵⁹

Thus, parenteral estrogens do not induce the same prothrombotic changes in coagulation factors that are seen with oral therapy, providing the biologic basis for the observations that parenteral administration of estrogen may avoid or reduce the thromboembolic toxicity seen with oral estrogens. The differing effects of oral versus parenteral estrogen on coagulation factors are summarized in Table 1.

Table 1. Differing Effects of Oral Versus Parenteral Estrogens on Coagulation Factors

TABLE 1
Differing Effects of Oral versus Parenteral
Estrogens on Coagulation Factors.

| | Oral estrogen | Parenteral estrogen |
|---|---|---|
| Factor VII | Increases | No change |
| Factor VIII activity | Increases | No change |
| Antithrombin III activity | Decreases | Decreases |
| F_{1.2} | Increases | No change |
| APC resistance | Increases | No change |
| Fibrinogen | Decreases | No change |

### Body Composition, Lipid and Glucose Metabolism

Metabolic and body compositional changes induced by ADT may predispose prostate cancer patients to a higher risk of cardiovascular disease over the long term. A prospective study evaluating the effect of ADT on lipids showed significant increases in total cholesterol, HDL, LDL, and serum triglyceride levels.¹¹ An increase in lipoprotein (a) (Lp(a)) levels has also been reported in prostate cancer patients after orchiectomy.⁷⁸

ADT also has been shown to adversely affect body composition, by increasing weight and percentage fat body mass, as well as decreasing percentage of lean body mass and muscle size.^{14,15} These adverse body compositional changes were associated with a rise in the augmentation of central arterial pressure, suggesting large artery stiffening.¹⁵ A positive correlation was noted between the change in fat mass and a rise in insulin concentration, suggesting reduced insulin sensitivity.¹⁵ This is notable in light of data on the metabolic syndrome, showing that excess fat in the central versus peripheral part of the body, independent of overall obesity, is associated with higher plasma glucose and insulin, hyperlipidemia, and decreased HDL cholesterol concentrations, components of the insulin resistance syndrome, and constituting a cluster of risk factors for atherosclerotic cardiovascular disease.⁷⁹ It has further been shown that prostate cancer patients with diabetes mellitus required more intensive antidiabetic therapy after ADT.¹³ Thus ADT appears to induce hormonal and body compositional changes that potentially lead to development of the metabolic syndrome, putting patients at risk of long-term cardiovascular complications.

In contrast, the use of estrogen may avoid the unfavorable metabolic changes induced by ADT. Treatment of men with prostate cancer with oral estrogen has been shown to lead to improvements in lipid profiles, including elevation of HDL-c and reductions in total and LDL cholesterol, as well as Lp(a).^{78,80-82} An unfavorable observation with oral estrogen, however, is an increase in triglyceride levels,^{83,84} higher levels of which have been associated with increased risk for mortality.⁸⁵ On the other hand, parenteral estrogen in men with prostate cancer has shown the same benefits on LDL and HDL levels without increasing triglyceride levels.^{86,87} This is in contrast to the unfavorable lipid profile induced by ADT.

A study found similarly that treatment of men on conventional ADT with transdermal estrogen resulted in significant decreases in total cholesterol, LDL cholesterol, and apolipoprotein B, without increases in triglyceride and Lp(a) levels. HDL cholesterol increased on therapy primarily as a result of a significant increase in HDL₂ particles. Further, there was no induction of the inflammatory markers IL-6, HS-CRP, and TNF-alpha with transdermal estrogen therapy.⁸⁸ These favorable changes have the potential to translate into reductions in cardiovascular morbidity over the long term.

### Bone Turnover

Skeletal related events in men on ADT are a significant and growing problem.^{33,89} Sex steroids are necessary to maintain bone homeostasis in adults, and hypogonadism in men are associated with loss of bone mass.⁴¹ The first year of ADT results in a 5%-10% decrease in bone mineral density, an effect greater than that associated with female menopause.¹⁰ Large population-based studies show that ADT is associated with an approximately 50% increase in the risk of fracture and this effect is dose dependent.⁸⁹ In metastatic prostate cancer, the risk of osteoporotic fracture is even greater than the risk of pathological fracture.⁹⁰ Thus ADT causes substantial morbidity and possibly mortality by accelerating bone loss.

Estrogen may play a greater role than testosterone in maintaining bone density in men. Estrogen insensitivity and aromatase deficiency cause decreased bone mineral density (BMD) in men despite normal or elevated testosterone levels, and plasma concentrations of total estradiol and bioavailable estrogen are independently related to BMD in men.⁴¹ Osteoclasts and osteoblasts are known to express estrogen receptors. Estrogens both stimulate osteoblasts and decrease osteoclastic activity.³³ In contrast to standard ADT by orchiectomy or GnRH agonists, medical castration with estrogen does not appear to cause bone loss in men with prostate cancer.^{33,58,91} Oral DES treatment was associated with significantly lower urinary N-telopeptide levels, a marker for bone turnover, than conventional ADT in men with non-metastatic prostate cancer.⁹² Similarly, in contrast to conventional ADT, estrogen-based hormonal therapy was not associated with decreases in bone mineral density (BMD).⁹³ Indeed, in Ockrim's study with transdermal estradiol, BMD increased with therapy.^{58,91}

Therefore parenteral estrogen therapy appears to not only protect against bone density loss when compared to ADT, but to improve the bone mineral density in prostate cancer patients.

### Hot Flashes

Conventional ADT methods, including orchiectomy and GnRH agonists, are associated with hot flashes in more than 50% of men.³³ Hot flashes significantly affect quality of life in prostate cancer patients undergoing ADT and may not necessarily abate over the course of therapy.¹⁶ Severe hot flashes are incapacitating, resulting in the inability to do work or usual activity, and can interrupt sleep. While the relationship between hot flashes and sleep disturbance has not been specifically studied in men with prostate cancer, studies in women demonstrate nocturnal hot flashes are associated with sleep disturbance⁹⁴ and sleep disturbances have been associated with significant decreases in quality of life.⁹⁵

Both oral and transdermal estrogen have been shown to prevent or treat hot flashes in men who have received ADT for prostate cancer. In a randomized, placebo-controlled trial in orchiectomy patients, DES at a dose of 1 mg per day resulted in resolution of hot flashes in 86% of patients and significant improvement in the remaining 14%.⁹⁶ Similar results were reported with transdermal estrogen.⁷⁷ A phase II study with transdermal estrogen found a significant reduction in hot flashes at 8 weeks of treatment.⁵⁹ Improvements in hot flashes would be expected to produce better sleep quality, fatigue reduction, and better overall quality of life.

As described above, androgen deprivation therapy is used for treating prostate cancer. In particular, primary hormonal therapy for prostate cancer typically involves bilateral orchiectomy or medical castration with luteinizing hormone-releasing hormone agonists or antagonists. Other methods of primary hormonal therapy may include blockage of androgen receptors with specific receptor blockers, and inhibition of steroid hormone production by agents such as ketoconazole or aminoglutethamide. In certain embodiments, the adverse effects that arise during or after ADT can be ameliorated by transdermally administering DES as described above either as an adjuvant to the ADT, or in conjunction with the ADT. Administration of the transdermal DES may occur simultaneously with, or subsequent to, the ADT.

### Example 1

A topical cream or gel may be prepared by dissolving 20 g of poloxamer 188 (Pluronic F86; a block copolymer of ethylene oxide and propylene oxide) in water to make 100 mol of a 20% solution. A lecithin/isopropyl myristate solution may be made by adding 10 g of lecithin to 10 g of isopropyl myristate. DES can be added at a desired concentration (e.g., a range of 0.5% to 5% weight, based on the total weight of the gel) to the lecithin/isopropyl myristate solution. The poloxamer solution and the DES/lecithin/isopropyl myristate solution may be mixed together to make a poloxamer lecithin organogel. The resulting gel will have the consistency of a thick cream.

Heterozygous nude mice (BALB/c or equivalent) may be treated with the above-described topical DES cream one to three times per day for one to 5 days. Initially, dose ranging experiments may be performed to test drug levels of a range of concentrations (as above) after a single dose. Blood will be drawn 1 to 2 hours after a single application and DES concentrations will be measured by mass spectrometry. Once an optimal DES concentration is selected based on the initial dose ranging study, mice will be treated two to three times per day for 3 to 5 days and have blood drawn for DES and testosterone levels. DES levels will demonstrate drug absorption. Reduced testosterone levels will demonstrate a biologic effect relevant to prostate cancer treatment (since in certain embodiments, transdermal DES will be used as initial hormonal therapy where the mechanism of action is related to testosterone suppression). Bioavailability will be established by administering the same doses of DES by oral gavage and by tail vein injection and measuring blood concentrations.

Additional experiments in homozygous nude mice that carry either subcutaneously implanted or intraprostatically implanted human tumor cell (nude mouse xenografts) may be used to further demonstrate an anti-tumor effect. Any number of cell lines, such as LNCaP, can be used to establish these tumors. A description of the intraprostatic approach is available in Garzotto M, Huryk R, Fair W R, Heston W D. Prostatic tumor implantation in the nude mouse via a perineal approach. Prostate. 1997 Sep. 15; 33(1):60-3.

### Example 2

### PLO Gel

PLO gel was made by combining a 20% (g/ml) poloxamer solution of Pluronic F68 (5 g in 25 ml Milli-Q H₂0; Sigma, cat #P1300) with a 50/50 (g/g) solution of lecithin (Spectrum Chemical, cat #L1083) and isopropyl palmitate (Spectrum Chemical, cat #IS160) in a 1:4 ratio (5 ml and 20 ml, respectively). Prior to combining the two solutions, each was produced as above then allowed to stir overnight at room temperature on a stir plate. The poloxamer solution was clear and the lecithin solution brown; both were transparent and viscous.

375 mg of diethylstilbestrol (DES; Sigma, cat #D4628) was then added to 2.5 ml of the lecithin solution and combined with 10 ml of the 20% poloxamer solution. This combination was stirred overnight at room temperature. The result was a viscous 3% (g/ml) DES cream, of which 0.25 ml was applied to each mouse resulting in a topical dose of approximately 7.5 mg.

### Sample Analysis by TSQ-ESI-MS

100 μl samples of commercial BALB/C mouse plasma containing the following concentrations of DES were used to produce a standard curve: 0, 0.01, 0.02, 0.05, 0.08, 0.1, 0.2, 0.5, 0.8, 1.0 (nanograms of DES per milliliter of mouse plasma). Each plasma standard was spiked with 10 μl of d8-DES (Isotech, cat #614289) used as an internal standard. Prior to the next step, samples taken from live mice were exposed to 5 μl of β-glucuronidase (Sigma, cat #G7017) and incubated at 37° C. for 70 minutes at 100 rpm. The enzymatic reaction was necessary in order to separate the DES molecule from any conjugated glucoronic acid that may be present. Following this incubation, 10 μl of 6M perchloric acid and 50 μl of acetonitrile were added. Each sample was then mixed for 2 minutes, following by centrifugation at 17,000×g for 5 minutes at 5° C. The supernatant was combined with 40 μl of 0.75M potassium carbonate and mixed for 30 seconds. Precipitated potassium perchlorate was removed by centrifugation at 17,000×g for 5 minutes at 5° C. The resulting supernatant was subjected to TSQ-ESI-MS. The chromatography conditions were the following: 25 μl metal loop, 10 μl no waste injection; oven 22° C.; flow rate 0.3 ml/min; solvents: A=water, C=MeOH; column: Hypersil Gold 100×2.1 mm, 5 μm bore (Thermo Scientific, catalog #25005-102130; with guard column).

### Standard Curve Results

| | DES | d8-DES | Area | Specified | Calculated | % RSD- | | | |
|---|---|---|---|---|---|---|---|---|---|
| Sample | Area | Area | Ratio | Amount | Amount | AMT | Level | Units | RT |
| O ng/ml | 53203 | 76399449 | 0.001 | 0.000 | 0.000 | 0.0% | 0 | ng/ml | 3.43 |
| 0.01 ng/ml | 332561 | 70811544 | 0.005 | 0.010 | 0.012 | 0.0% | 0.01 | ng/ml | 3.39 |
| 0.02 ng/ml | 686962 | 91059917 | 0.008 | 0.020 | 0.020 | 8.2% | 0.02 | ng/ml | 3.39 |
| 0.02 ng/ml | 557227 | 82032181 | 0.007 | 0.020 | 0.018 | 8.2% | 0.02 | ng/ml | 3.40 |
| 0.05 ng/ml | 1390012 | 83120151 | 0.017 | 0.050 | 0.048 | 0.0% | 0.05 | ng/ml | 3.36 |
| 0.05 ng/ml | 1534148 | 91776159 | 0.017 | 0.050 | 0.048 | 0.0% | 0.05 | ng/ml | 3.36 |
| 0.08 ng/ml | 2493917 | 87153125 | 0.029 | 0.080 | 0.083 | 0.0% | 0.08 | ng/ml | 3.36 |
| 0.1 ng/ml | 2943330 | 90163318 | 0.033 | 0.100 | 0.095 | 0.0% | 0.1 | ng/ml | 3.33 |
| 0.2 ng/ml | 5453295 | 92314688 | 0.059 | 0.200 | 0.174 | 0.0% | 0.2 | ng/ml | 3.36 |
| 0.5 ng/ml | 14633562 | 91096858 | 0.161 | 0.500 | 0.476 | 0.0% | 0.5 | ng/ml | 3.33 |
| 0.8 ng/ml | 24577470 | 86697839 | 0.283 | 0.800 | 0.842 | 0.0% | 0.8 | ng/ml | 3.33 |
| 1.0 ng/ml | 27802138 | 81444773 | 0.341 | 1.000 | 1.014 | 0.0% | 1.0 | ng/ml | 3.33 |
| blk | NF | 6185 | NF | NA | NF | NF | NA | ng/ml | NF |
| enzyme | NF | 386 | NF | NA | NF | NF | NA | ng/ml | NF |

### Mouse Experiment #1

As a positive control, 75 mg of DES was dissolved in 1 ml dimethyl sulfoxide (DMSO). 10 μl of this solution was combined with 90 μl of normal saline, and injected in Mouse 1 i.p. A cardiac puncture was performed under 3% isoflurane 1 hour following the injection. The final dose was 0.75 mg.

Mice 2, 3, and 4 were each shaved under 1.5% isoflurane, then 0.25 ml of the 3% DES cream was applied dorsally (approximately 2 square inches). Cardiac punctures were performed on mice 2, 3, and 4 at 1, 4, and 7 hours, respectively. Each cardiac puncture sample was combined with 0.1 ml sodium heparin (50 mg/ml; Sigma, cat #).

Blood plasma was isolated from each sample by centrifugation at 4,000 rpm for 15 minutes. This yielded approximately 0.2 to 0.4 ml of plasma from each sample.

### DES Results

| | Volume | | | | DES | d8-DES | Area | | | |
|---|---|---|---|---|---|---|---|---|---|---|
| Sample | (μl) | Treatment | Timepoint | Sample Prep | Area | Area | Ratio | Amount | Units | RT |
| 1 | 100 | DES i.p. | 1 hour | — | 42082 | 1376354 | 0.031 | 0.089 | ng/ml | 3.19 |
| 1 | 100 | DES i.p. | 1 hour | β-glucuronidase | 23802212 | 47723043 | 0.499 | 1.483 | ng/ml | 3.30 |
| 2 | 50 | 0.25 ml Cream | 1 hour | — | 219514 | 74304860 | 0.003 | 0.007 | ng/ml | 3.29 |
| 2 | 50 | 0.25 ml Cream | 1 hour | β-glucuronidase | 194708 | 24046180 | 0.008 | 0.022 | ng/ml | 3.09 |
| 3 | 100 | 0.25 ml Cream | 4 hours | — | 84585 | 23589970 | 0.004 | 0.009 | ng/ml | 3.33 |
| 3 | 100 | 0.25 ml Cream | 4 hours | β-glucuronidase | 2676063 | 41695410 | 0.064 | 0.189 | ng/ml | 3.26 |
| 4 | 100 | 0.25 ml Cream | 7 hours | — | 26495 | 13095766 | 0.002 | 0.004 | ng/ml | 3.22 |
| 4 | 100 | 0.25 ml Cream | 7 hours | β-glucuronidase | 2288285 | 44308485 | 0.052 | 0.152 | ng/ml | 3.26 |

The results of this experiment show that at the 4 and 7 hour timepoints >1% of the DES from the cream application was recovered in the mouse plasma following the exposure to β-glucuronidase.

### Mouse Experiment #2

Six athymic nude mice (Harlan) were used for this experiment. As above, mouse 1 received a 100 μl injection of a 7.5% DES solution in DMSO, for a total dose of 0.75 mg. Mice 2, 3, 4, 5, and 6 received three applications of 0%, 0.5%, 1%, 2%, and 3% DES cream at 24 h intervals. 24 h following the final cream application and 1 h post-injection, blood was taken from each mouse by cardiac puncture. Each cardiac puncture sample was combined with 0.1 ml sodium heparin (50 mg/ml; Sigma, cat #). Blood plasma was isolated from each sample by centrifugation at 4,000 rpm for 15 minutes. This yielded approximately 0.2 to 0.4 ml of plasma from each sample.

### DES Results

| | Volume | | | | DES | d8-DES | Area | | | |
|---|---|---|---|---|---|---|---|---|---|---|
| Sample | (1J1I | Treatment | Timepoint | Sample Prep | Area | Area | Ratio | Amount | Units | RT |
| 1 | 100 | DES i.p. | 1 hour | β-glucuronidase | 31760633 | 53216731 | 0.597 | 1.926 | ng/ml | 5.72 |
| 2 | 250 | 0% DES Cream | 72 hrs | β-glucuronidase | 148790 | 68246991 | 0.002 | 0 | ng/ml | 5.72 |
| 3 | 250 | 0.5% DES Cream | 72 hrs | β-glucuronidase | 906962 | 52355518 | 0.017 | 0.054 | ng/ml | 5.68 |
| 4 | 250 | 1% DES Cream | 72 hrs | β-glucuronidase | 2485371 | 55747891 | 0.045 | 0.142 | ng/ml | 5.68 |
| 5 | 250 | 2% DES Cream | 72 hrs | β-glucuronidase | 6755534 | 60205536 | 0.112 | 0.360 | ng/ml | 5.71 |
| 6 | 250 | 3% DES Cream | 72 hrs | β-glucuronidase | 2355590 | 40568121 | 0.058 | 0.185 | ng/ml | 5.71 |

### Testosterone Results

A radioimmunoassay that detects both dihydrotestosterone and testosterone was used to assess androgen suppression by DES.

| | Volume | | | [testosterone] |
|---|---|---|---|---|
| Sample | (1J1I | Treatment | Timepoint | (pg/mlI |
| 1 | 100 | DES i.p. | 1 hour | 143 |
| 2 | 250 | 0% DES Cream | 72 hrs | — |
| 3 | 250 | 0.5% DES Cream | 72 hrs | 54.7 |
| 4 | 250 | 1% DES Cream | 72 hrs | 47.1 |
| 5 | 250 | 2% DES Cream | 72 hrs | — |
| 6 | 250 | 3% DES Cream | 72 hrs | 76.6 |

Not enough blood was obtained from the 0 and 2% DES treated mice to allow for androgen measurement. However, using the injected mouse as a baseline, testosterone suppression was observed in those receiving 0.5, 1, or 3% DES cream.

The data from Example 2 demonstrates administering DES transdermally, that generally higher concentrations of DES are achievable with increasing concentrations of DES in the gel preparation, and that transdermal DES can suppress serum testosterone.

The methods disclosed herein are described in more detail in the following numbered paragraphs:

1. A method for treating prostate cancer comprising transdermally administering a therapeutically effective amount of diethylstilbesterol, or a pharmaceutically acceptable salt or complex thereof, to a subject.

2. The method of paragraph 1, wherein the diethylstilbesterol is administered via a transdermal cream, lotion, gel, suspension or ointment.

3. The method of paragraph 1, wherein the diethylstilbesterol is administered via a transdermal patch.

4. The method of any one of paragraphs 1-3, wherein the subject has been, or is, also subjected to androgen deprivation therapy.

5. The method of any one of paragraphs 1-4, wherein about 0.1 to about 25 mg/day diethylstilbesterol is administered.

6. The method of any one of paragraphs 1-4, wherein about 1 to about 5 mg/day diethylstilbesterol is administered.

7. The method of any one of paragraphs 1-4, wherein about 0.5 to about 10 mg/day diethylstilbesterol is administered.

8. The method of paragraph 2, wherein the transdermal cream, lotion, gel, suspension or ointment includes about 0.5% to about 5% by weight diethylstilbesterol.

9. The method of any one of paragraphs 1-8, wherein the subject is contraindicated for androgen deprivation therapy.

10. The method of any of paragraphs 1-9, wherein the prostate cancer is symptomatic locally advanced prostate cancer.

11. The method of any one of paragraphs 1-9, wherein the prostate cancer is metastatic prostate cancer.

12. A method for treating hot flashes in a castrate prostate cancer patient, comprising transdermally administering a therapeutically effective amount of diethylstilbesterol, or a pharmaceutically acceptable salt or complex thereof, to the patient.

13. The method of paragraph 12, wherein the diethylstilbesterol is administered via a transdermal cream, lotion, gel, suspension or ointment.

14. The method of paragraph 12, wherein the diethylstilbesterol is administered via a transdermal patch.

15. The method of any one of paragraphs 12-14, wherein about 0.1 to about 25 mg/day diethylstilbesterol is administered.

16. The method of any one of paragraphs 12-14, wherein about 1 to about 5 mg/day diethylstilbesterol is administered.

17. The method of any one of paragraphs 12-14, wherein about 0.5 to about 10 mg/day diethylstilbesterol is administered.

18. The method of paragraph 13, wherein the transdermal cream, lotion, gel, suspension or ointment includes about 0.5% to about 5% by weight diethylstilbesterol.

19. The method of any one of paragraphs 12-18, wherein the subject has been, or is, also subjected to androgen deprivation therapy.

20. A method for treating osteoporosis in a castrate prostate cancer patient, comprising transdermally administering a therapeutically effective amount of diethylstilbesterol, or a pharmaceutically acceptable salt or complex thereof, to the patient.

21. The method of paragraph 20, wherein the diethylstilbesterol is administered via a transdermal cream, lotion, gel, suspension or ointment.

22. The method of paragraph 20, wherein the diethylstilbesterol is administered via a transdermal patch.

23. The method of any one of paragraphs 20-22, wherein about 0.1 to about 25 mg/day diethylstilbesterol is administered.

24. The method of any one of paragraphs 20-22, wherein about 1 to about 5 mg/day diethylstilbesterol is administered.

25. The method of any one of paragraphs 20-22, wherein about 0.5 to about 10 mg/day diethylstilbesterol is administered.

26. The method of paragraph 21, wherein the transdermal cream, lotion, gel, suspension or ointment includes about 0.5% to about 5% by weight diethylstilbesterol.

27. The method of any one of paragraphs 20-26, wherein the subject has been, or is, also subjected to androgen deprivation therapy.

28. A method for treating a taxane-resistant disease comprising transdermally administering a therapeutically effective amount of diethylstilbesterol, or a pharmaceutically acceptable salt or complex thereof, to a subject who has, or is diagnosed with, a taxane-resistant disease.

### CITATIONS

1. Wasson J H, Fowler F J, Jr., Barry M J: Androgen deprivation therapy for asymptomatic advanced prostate cancer in the prostate specific antigen era: a national survey of urologist beliefs and practices. J Urol 159:1993-6; discussion 1996-7, 1998
2. The Medical Research Council Prostate Cancer Working Party Investigators Group. Immediate versus deferred treatment for advanced prostatic cancer: initial results of the Medical Research Council Trial. Br J Urol 79:235-46, 1997
3. Loblaw D A, Mendelson D S, Talcott J A, et al: American society of clinical oncology recommendations for the initial hormonal management of androgen-sensitive metastatic, recurrent, or progressive prostate cancer. J Clin Oncol 22:2927-41, 2004
4. Cooperberg M R, Grossfeld G D, Lubeck D P, et al: National practice patterns and time trends in androgen ablation for localized prostate cancer. J Natl Cancer Inst 95:981-9, 2003
5. Messing E M, Manola J, Sarosdy M, et al: Immediate hormonal therapy compared with observation after radical prostatectomy and pelvic lymphadenectomy in men with node-positive prostate cancer. N Engl J Med 341:1781-8, 1999
6. Pilepich M V, Winter K, John M J, et al: Phase III radiation therapy oncology group (RTOG) trial 86-10 of androgen deprivation adjuvant to definitive radiotherapy in locally advanced carcinoma of the prostate. Int J Radiat Oncol Biol Phys 50:1243-52, 2001
7. Bolla M, Gonzalez D, Warde P, et al: Improved survival in patients with locally advanced prostate cancer treated with radiotherapy and goserelin. N Engl J Med 337:295-300, 1997
8. Salminen E K, Wickstrom J E, Vahlberg T, et al: Trends in the use of androgen deprivation in prostate cancer. Acta Oncol 43:382-7, 2004
9. Janoff D M, Peterson C, Mongoue-Tchokote S, et al: Clinical Outcomes of Androgen Deprivation as Sole Therapy for Localized and Locally Advanced Prostate Cancer. BJU Int in press, 2005
10. Smith M R: Osteoporosis during androgen deprivation therapy for prostate cancer. Urology 60:79-85; discussion 86, 2002
11. Smith M R: Osteoporosis and other adverse body composition changes during androgen deprivation therapy for prostate cancer. Cancer Metastasis Rev 21:159-66, 2002
12. Nowicki M, Bryc W, Kokot F: Hormonal regulation of appetite and body mass in patients with advanced prostate cancer treated with combined androgen blockade. J Endocrinol Invest 24:31-6, 2001
13. Fukui M, Koyama M, Nakagawa Y, et al: Castration and diabetes. Diabetes Care 23:1032-3, 2000
14. Smith M R, Finkelstein J S, McGovern F J, et al: Changes in body composition during androgen deprivation therapy for prostate cancer. J Clin Endocrinol Metab 87:599-603, 2002
15. Smith J C, Bennett S, Evans L M, et al: The effects of induced hypogonadism on arterial stiffness, body composition, and metabolic parameters in males with prostate cancer. J Clin Endocrinol Metab 86:4261-7, 2001
16. Higano C S: Side effects of androgen deprivation therapy: monitoring and minimizing toxicity. Urology 61:32-8, 2003
17. Herr H W, O'Sullivan M: Quality of life of asymptomatic men with nonmetastatic prostate cancer on androgen deprivation therapy. J Urol 163:1743-6, 2000
18. Huggins C, Hodges C V: Studies on prostatic cancer: I. The effect of castration, of estrogen and of androgen injection on serum phosphatases in metastatic carcinoma of the prostate. Cancer Res 1:293-297, 1941
19. Aronson N, Seidenfeld J, Samson D, et al: Relative Effectiveness and Cost-Effectiveness of Methods of Androgen Suppression in the Treatment of Advanced Prostate Cancer. Evidence Report/Technology Assessment No. 4. Agency for Health Care Policy and Research Publication No. 99-E0012, 1999
20. Oh W K: The evolving role of estrogen therapy in prostate cancer. Clin Prostate Cancer 1:81-9, 2002
21. Whitesel J A: The case for diethylstilbestrol. J Urol 169:290-291, 2003
22. Malkowicz S B: The role of diethylstilbestrol in the treatment of prostate cancer. Urology 58:108-13, 2001
23. Byar D P: Proceedings: The Veterans Administration Cooperative Urological Research Group's studies of cancer of the prostate. Cancer 32:1126-30, 1973
24. Kent J R, Bischoff A J, Arduino L J, et al: Estrogen dosage and suppression of testosterone levels in patients with prostatic carcinoma. J Urol 109:858-60, 1973
25. Hedlund P O, Ala-Opas M, Brekkan E, et al: Parenteral estrogen versus combined androgen deprivation in the treatment of metastatic prostatic cancer—Scandinavian Prostatic Cancer Group (SPCG) Study No. 5. Scand J Urol Nephrol 36:405-13, 2002
26. Henriksson P, Carlstrom K, Pousette A, et al: Time for revival of estrogens in the treatment of advanced prostatic carcinoma? Pharmacokinetics, and endocrine and clinical effects, of a parenteral estrogen regimen. Prostate 40:76-82, 1999
27. Robertson C N, Roberson K M, Padilla G M, et al: Induction of apoptosis by diethylstilbestrol in hormone-insensitive prostate cancer cells. J Natl Cancer Inst 88:908-17, 1996
28. El Etreby M F, Liang Y, Lewis R W: Induction of apoptosis by mifepristone and tamoxifen in human LNCaP prostate cancer cells in culture. Prostate 43:31-42, 2000
29. Ho S M: Estrogens and anti-estrogens: key mediators of prostate carcinogenesis and new therapeutic candidates. J Cell Biochem 91:491-503, 2004
30. Schulz P, Bauer H W, Brade W P, et al: Evaluation of the cytotoxic activity of diethylstilbestrol and its mono- and diphosphate towards prostatic carcinoma cells. Cancer Res 48:2867-70, 1988
31. Kim H T, Kim B C, Kim I Y, et al: Raloxifene, a mixed estrogen agonist/antagonist, induces apoptosis through cleavage of BAD in TSU-PR1 human cancer cells. J Biol Chem 277:32510-5, 2002
32. Mobley J A, L'Esperance J O, Wu M, et al: The novel estrogen 17alpha-20Z-21-[(4-amino)phenyl]-19-norpregna-1,3,5(10),20-tetraene-3,17beta-diol induces apoptosis in prostate cancer cell lines at nanomolar concentrations in vitro. Mol Cancer Ther 3:587-95, 2004
33. Scherr D S, Pitts W R, Jr.: The nonsteroidal effects of diethylstilbestrol: the rationale for androgen deprivation therapy without estrogen deprivation in the treatment of prostate cancer. J Urol 170:1703-8, 2003
34. Corey E, Quinn J E, Emond M J, et al: Inhibition of androgen-independent growth of prostate cancer xenografts by 17beta-estradiol. Clin Cancer Res 8:1003-7, 2002
35. Lakhani N J, Sarkar M A, Venitz J, et al: 2-Methoxyestradiol, a promising anticancer agent. Pharmacotherapy 23:165-72, 2003
36. Qadan L R, Perez-Stable C M, Anderson C, et al: 2-Methoxyestradiol induces G2/M arrest and apoptosis in prostate cancer. Biochem Biophys Res Commun 285:1259-66, 2001
37. Kumar A P, Garcia G E, Slaga T J: 2-methoxyestradiol blocks cell-cycle progression at G(2)/M phase and inhibits growth of human prostate cancer cells. Mol Carcinog 31:111-24, 2001
38. Mabjeesh N J, Escuin D, LaVallee T M, et al: 2ME2 inhibits tumor growth and angiogenesis by disrupting microtubules and dysregulating HIF. Cancer Cell 3:363-75, 2003
39. Wilding G, Sweeney C, King D M, et al: Phase 2, multicenter, randomized, double blind, safety, pharmacokinetic, pharmacodynamic and efficacy study of two doses of 2-methoxyestradiol administered orally in patients with hormone refractory prostate cancer. Abstract No. 338., 2002 ASCO Annual Meeting. Orlando, Fla., 2002
40. Kuiper G G, Enmark E, Pelto-Huikko M, et al: Cloning of a novel receptor expressed in rat prostate and ovary. Proc Natl Acad Sci USA 93:5925-30, 1996
41. de Ronde W, Pois H, van Leeuwen J, et al: The importance of oestrogens in males. Clin Endocrinol (Oxf) 58:529-542, 2003
42. Horvath L G, Henshall S M, Lee C S, et al: Frequent loss of estrogen receptor-beta expression in prostate cancer. Cancer Res 61:5331-5, 2001
43. Linja M J, Savinainen K J, Tammela T L, et al: Expression of ERalpha and ERbeta in prostate cancer. Prostate 55:180-6, 2003
44. Maggiolini M, Grazia Recchia A, Carpino A, et al: Oestrogen receptor beta is required for androgen-stimulated proliferation of LNCaP prostate cancer cells. J Mol Endocrinol 32:777-91, 2004
45. de Voogt H J, Smith P H, Pavone-Macaluso M, et al: Cardiovascular side effects of diethylstilbestrol, cyproterone acetate, medroxyprogesterone acetate and estramustine phosphate used for the treatment of advanced prostatic cancer: results from European Organization for Research on Treatment of Cancer trials 30761 and 30762. J Urol 135:303-7, 1986
46. Robinson M R, Smith P H, Richards B, et al: The final analysis of the EORTC Genito-Urinary Tract Cancer Co-Operative Group phase III clinical trial (protocol 30805) comparing orchidectomy, orchidectomy plus cyproterone acetate and low dose stilboestrol in the management of metastatic carcinoma of the prostate. Eur Urol 28:273-83, 1995
47. Shearer R J, Hendry W F, Ferguson J D: Oestrogen treatment in carcinoma of the prostate. Br Med J 3:51, 1973
48. Bishop M C: Experience with low-dose oestrogen in the treatment of advanced prostate cancer: a personal view. Br J Urol 78:921-7; discussion 927-8, 1996
49. Leuprolide versus diethylstilbestrol for metastatic prostate cancer. The Leuprolide Study Group. N Engl J Med 311:1281-6, 1984
50. Glashan R W, Robinson M R: Cardiovascular complications in the treatment of prostatic carcinoma. Br J Urol 53:624-7, 1981
51. Klotz L, McNeill I, Fleshner N: A phase 1-2 trial of diethylstilbestrol plus low dose warfarin in advanced prostate carcinoma. J Urol 161:169-72, 1999
52. Chang A, Yeap B, Davis T, et al: Double-blind, randomized study of primary hormonal treatment of stage D2 prostate carcinoma: flutamide versus diethylstilbestrol. J Clin Oncol 14:2250-7, 1996
53. Hedlund P O, Henriksson P: Parenteral estrogen versus total androgen ablation in the treatment of advanced prostate carcinoma: effects on overall survival and cardiovascular mortality. The Scandinavian Prostatic Cancer Group (SPCG)-5 Trial Study. Urology 55:328-33, 2000
54. Daehlin L, Damber J E, von Schoultz B, et al: The oestrogenic effects of ethinyl oestradiol/polyoestradiol phosphate and estramustine phosphate in patients with prostatic carcinoma. A comparative study of oestrogen sensitive liver proteins, gonadotrophins and prolactin. Br J Urol 58:412-6, 1986
55. Henriksson P, Blomback M, Eriksson A, et al: Effect of parenteral oestrogen on the coagulation system in patients with prostatic carcinoma. Br J Urol 65:282-5, 1990
56. Henriksson P, Eriksson A, Stege R, et al: Cardiovascular follow-up of patients with prostatic cancer treated with single-drug polyestradiol phosphate. Prostate 13:257-61, 1988
57. Henriksson P, Blomback M, Bratt G, et al: Effects of oestrogen therapy and orchidectomy on coagulation and prostanoid synthesis in patients with prostatic cancer. Med Oncol Tumor Pharmacother 6:219-25, 1989
58. Ockrim J L, Lalani E N, Laniado M E, et al: Transdermal estradiol therapy for advanced prostate cancer—forward to the past? J Urol 169:1735-7, 2003
59. Bland L B, Garzotto M, Deloughery T G, et al: Phase II study of transdermal estradiol in androgen-independent prostate carcinoma. Cancer 103:717-23, 2005
60. Cushman M, Meilahn E N, Psaty B M, et al: Hormone replacement therapy, inflammation, and hemostasis in elderly women. Arterioscler Thromb Vasc Biol 19:893-9, 1999
61. Dobbs R M, Barber J A, Weigel J W, et al: Clotting predisposition in carcinoma of the prostate. J Urol 123:706-9, 1980
62. Kroon U B, Silfverstolpe G, Tengborn L: The effects of transdermal estradiol and oral conjugated estrogens on haemostasis variables. Thromb Haemost 71:420-3, 1994
63. Aro J, Haapiainen R, Rasi V, et al: The effect of parenteral estrogen versus orchiectomy on blood coagulation and fibrinolysis in prostatic cancer patients. Eur Urol 17:161-5, 1990
64. Baker V L: Alternatives to oral estrogen replacement. Transdermal patches, percutaneous gels, vaginal creams and rings, implants, other methods of delivery. Obstet Gynecol Clin North Am 21:271-97, 1994
65. Basbug M, Aygen E, Tayyar M, et al: Twenty two weeks of transdermal estradiol increases sex hormone-binding globulin in surgical menopausal women. Eur J Obstet Gynecol Reprod Biol 73:149-52, 1997
66. Mashchak C A, Lobo R A, Dozono-Takano R, et al: Comparison of pharmacodynamic properties of various estrogen formulations. Am J Obstet Gynecol 144:511-8, 1982
67. Steingold K A, Cefalu W, Pardridge W, et al: Enhanced hepatic extraction of estrogens used for replacement therapy. J Clin Endocrinol Metab 62:761-6, 1986
68. Stomati M, Hartmann B, Spinetti A, et al: Effects of hormonal replacement therapy on plasma sex hormone-binding globulin, androgen and insulin-like growth factor-1 levels in postmenopausal women. J Endocrinol Invest 19:535-41, 1996
69. O'Connell M B: Pharmacokinetic and pharmacologic variation between different estrogen products. J Clin Pharmacol 35:18 S-24S, 1995
70. Scarabin P Y, Oger E, Plu-Bureau G: Differential association of oral and transdermal oestrogen-replacement therapy with venous thromboembolism risk. Lancet 362:428-32, 2003
71. Stevenson J C: The metabolic basis for the effects of HRT on coronary heart disease. Endocrine 24:239-44, 2004
72. Zegura B, Keber I, Sebestjen M, et al: Double blind, randomized study of estradiol replacement therapy on markers of inflammation, coagulation and fibrinolysis. Atherosclerosis 168:123-9, 2003
73. Selby P, McGarrigle H H, Peacock M: Comparison of the effects of oral and transdermal oestradiol administration on oestrogen metabolism, protein synthesis, gonadotrophin release, bone turnover and climacteric symptoms in postmenopausal women. Clin Endocrinol (Oxf) 30:241-9, 1989
74. Balfour J A, Heel R C: Transdermal estradiol. A review of its pharmacodynamic and pharmacokinetic properties, and therapeutic efficacy in the treatment of menopausal complaints. Drugs 40:561-82, 1990
75. Lowe G D, Upton M N, Rumley A, et al: Different effects of oral and transdermal hormone replacement therapies on factor IX, APC resistance, t-PA, PAI and C-reactive protein—a cross-sectional population survey. Thromb Haemost 86:550-6, 2001
76. Chetkowski R J, Meldrum D R, Steingold K A, et al: Biologic effects of transdermal estradiol. N Engl J Med 314:1615-20, 1986
77. Gerber G S, Zagaja G P, Ray P S, et al: Transdermal estrogen in the treatment of hot flushes in men with prostate cancer. Urology 55:97-101, 2000
78. Henriksson P, Angelin B, Berglund L: Hormonal regulation of serum Lp (a) levels. Opposite effects after estrogen treatment and orchidectomy in males with prostatic carcinoma. J Clin Invest 89:1166-71, 1992
79. Wajchenberg B L: Subcutaneous and visceral adipose tissue: their relation to the metabolic syndrome. Endocr Rev 21:697-738, 2000
80. Wallentin L, Varenhorst E: Plasma lipoproteins during anti-androgen treatment by estrogens or orchidectomy in men with prostatic carcinoma. Horm Metab Res 13:293-7, 1981
81. Agardh C D, Nilsson-Ehle P, Lundgren R, et al: The influence of treatment with estrogens and estramustine phosphate on platelet aggregation and plasma lipoproteins in non-disseminated prostatic carcinoma. J Urol 132:1021-4, 1984
82. Hiraga T, Shimokawa K, Murase T, et al: Reduction of serum lipoprotein (a) by estrogen in men with prostatic cancer. Endocr J 40:507-13, 1993
83. Wallentin L, Varenhorst E: Changes of plasma lipid metabolism in males during estrogen treatment for prostatic carcinoma. J Clin Endocrinol Metab 47:596-9, 1978
84. Usui S, Suzuki K, Yamanaka H, et al: Estrogen treatment of prostate cancer increases triglycerides in lipoproteins as demonstrated by HPLC and immunoseparation techniques. Clin Chim Acta 317:133-43, 2002
85. Seal U S, Doe R P, Byar D P, et al: Response of serum cholesterol and triglycerides to hormone treatment and the relation of pretreatment values to mortality in patients with prostatic cancer. Cancer 38:1095-107, 1976
86. Steg A, Benoit G: Percutaneous 17 beta-estradiol in treatment of cancer of prostate. Urology 14:373-5, 1979
87. Berglund L, Carlstrom K, Stege R, et al: Hormonal regulation of serum lipoprotein (a) levels: effects of parenteral administration of estrogen or testosterone in males. J Clin Endocrinol Metab 81:2633-7, 1996
88. Purnell J Q, Bland L B, Garzotto M, et al: Transdermal estrogen therapy improves cholesterol levels and lipid profiles in men with prostate cancer. Journal of Clinical Oncology, 2004 ASCO Annual Meeting Proceedings (Post-Meeting Edition) 22:8035, 2004
89. Shahinian V B, Kuo Y F, Freeman J L, et al: Risk of fracture after androgen deprivation for prostate cancer. N Engl J Med 352:154-64, 2005
90. Ross R W, Small E J: Osteoporosis in men treated with androgen deprivation therapy for prostate cancer. J Urol 167:1952-6, 2002
91. Ockrim J L, Lalani E N, Banks L M, et al: Transdermal Estradiol Improves Bone Density When Used as Single Agent Therapy for Prostate Cancer. J Urol 172:2203-2207, 2004
92. Scherr D, Pitts W R, Jr., Vaughn E D, Jr.: Diethylstilbesterol revisited: androgen deprivation, osteoporosis and prostate cancer. J Urol 167:535-8, 2002
93. Eriksson S, Eriksson A, Stege R, et al: Bone mineral density in patients with prostatic cancer treated with orchidectomy and with estrogens. Calcif Tissue Int 57:97-9, 1995
94. Savard J, Davidson J R, Ivers H, et al: The association between nocturnal hot flashes and sleep in breast cancer survivors. J Pain Symptom Manage 27:513-22, 2004
95. Schubert C R, Cruickshanks K J, Dalton D S, et al: Prevalence of sleep problems and quality of life in an older population. Sleep 25:889-93, 2002
96. Atala A, Amin M, Harty J I: Diethylstilbestrol in treatment of postorchiectomy vasomotor symptoms and its relationship with serum follicle-stimulating hormone, luteinizing hormone, and testosterone. Urology 39:108-10, 1992
97. Arduino L J, Bailar J C, 3rd, Becker L E, et al: Carcinoma of the Prostate: Treatment Comparions. The Veterans Administration Cooperative Urological Research Group. J Urol 98:516-22, 1967
98. Johansson J E, Andersson S O, Holmberg L, et al: Primary orchiectomy versus estrogen therapy in advanced prostatic cancer—a randomized study: results after 7 to 10 years of followup. J Urol 145:519-22; discussion 522-3, 1991
99. Aro J: Cardiovascular and all-cause mortality in prostatic cancer patients treated with estrogens or orchiectomy as compared to the standard population. Prostate 18:131-7, 1991
100. Lukkarinen O, Kontturi M: Comparison of a long-acting LHRH agonist and polyoestradiol phosphate in the treatment of advanced prostatic carcinoma. An open prospective, randomized multicentre study. Scand J Urol Nephrol 28:171-8, 1994
101. Mikkola A K, Ruutu M L, Aro J L, et al: Parenteral polyoestradiol phosphate vs orchidectomy in the treatment of advanced prostatic cancer. Efficacy and cardiovascular complications: a 2-year follow-up report of a national, prospective prostatic cancer study. Finnprostate Group. Br J Urol 82:63-8, 1998

What is claimed is:

1. A method for treating prostate cancer comprising transdermally administering a therapeutically effective amount of diethylstilbesterol, or a pharmaceutically acceptable salt or complex thereof, to a subject.

2. The method of claim 1, wherein the diethylstilbesterol is administered via a transdermal cream, lotion, gel, suspension or ointment.

3. The method of claim 1, wherein the diethylstilbesterol is administered via a transdermal patch.

4. The method of claim 1, wherein the subject has been, or is, also subjected to androgen deprivation therapy.

5. The method of claim 1, wherein about 0.1 to about 25 mg/day diethylstilbesterol is administered.

6. The method of claim 1, wherein about 1 to about 5 mg/day diethylstilbesterol is administered.

7. The method of any claim 1, wherein about 0.5 to about 10 mg/day diethylstilbesterol is administered.

8. The method of claim 2, wherein the transdermal cream, lotion, gel, suspension or ointment includes about 0.5% to about 5% by weight diethylstilbesterol.

9. The method of claim 1, wherein the subject is contraindicated for androgen deprivation therapy.

10. The method of any of claim 1, wherein the prostate cancer is symptomatic locally advanced prostate cancer.

11. The method of any one of claim 1, wherein the prostate cancer is metastatic prostate cancer.

12. A method for treating hot flashes in a castrate prostate cancer patient, comprising transdermally administering a therapeutically effective amount of diethylstilbesterol, or a pharmaceutically acceptable salt or complex thereof, to the patient.

13. The method of claim 12, wherein the diethylstilbesterol is administered via a transdermal cream, lotion, gel, suspension or ointment.

14. The method of claim 12, wherein the diethylstilbesterol is administered via a transdermal patch.

15. The method of claim 12, wherein about 0.1 to about 25 mg/day diethylstilbesterol is administered.

16. A method for treating osteoporosis in a castrate prostate cancer patient, comprising transdermally administering a therapeutically effective amount of diethylstilbesterol, or a pharmaceutically acceptable salt or complex thereof, to the patient.

17. The method of claim 16, wherein the diethylstilbesterol is administered via a transdermal cream, lotion, gel, suspension or ointment.

18. The method of claim 16, wherein the diethylstilbesterol is administered via a transdermal patch.

19. The method of claim 16, wherein about 0.1 to about 25 mg/day diethylstilbesterol is administered.

20. A method for treating a taxane-resistant disease comprising transdermally administering a therapeutically effective amount of diethylstilbesterol, or a pharmaceutically acceptable salt or complex thereof, to a subject who has, or is diagnosed with, a taxane-resistant disease.
